(19) United States
(12) Plant Patent Application Publication
(10) Pub. No.: US 2003/0009808 P1
(43) Pub. Date: Jan. 9, 2003

(54) Chrysanthemum plant named 'POWELL'

(75) Inventor: Robert Noodelijk, Woubrugge (NL)

Correspondence Address: PARKHURST & WENDEL, L.L.P., 1421 PRINCE STREET, SUITE 210, ALEXANDRIA, VA, 22314-2805, US

(73) Assignee: Chrysanthemum Breeders Association N.V.

(21) Appl. No.: 10/175,868

(22) Filed: Jun. 21, 2002

(30) Foreign Application Priority Data
Jun. 28, 2001 (NL) CHR3270

(51) Int. Cl. A01H 5/00

(52) U.S. Cl. PLT/295

(57) ABSTRACT

A Chrysanthemum plant named ‘POWELL’ characterized by its large sized blooms with huge yellow ray-florets and green disc florets.

### BACKGROUND OF THE INVENTION

‘POWELL’ is a product of a breeding-program which had the objective of creating new chrysanthemum cultivars with a daust type flower, a 5 week response and a medium plant height. The new plant of the present invention comprises a new and distinct cultivar of Chrysanthemum plant. ‘POWELL’ is a seedling from a cross in a breeding program maintained under the control of inventor. The female parent is ‘Miramar’ (U.S.Plant Pat. No. 7469), the male parent is 94.1106, an unpatented, unamed seedling. The male parent is not available to inventor for description. A comparison with the female parent is made in this description. The new and distinct cultivar was discovered and selected as a flowering plant within the progeny of the stated cross by Rob Noodelijk in a controlled environment (greenhouse) in Rijsenhout Holland in April 1997. The first act of asexual reproduction of ‘POWELL’ was accomplished when vegetative cuttings were taken from the initial selection in June 1997 in a controlled environment in Rijsenhout Holland.

### SUMMARY OF THE INVENTION

The present invention is a new and distinct variety of chrysanthemum bearing large sized blooms with huge yellow ray-florets and green disc florets.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

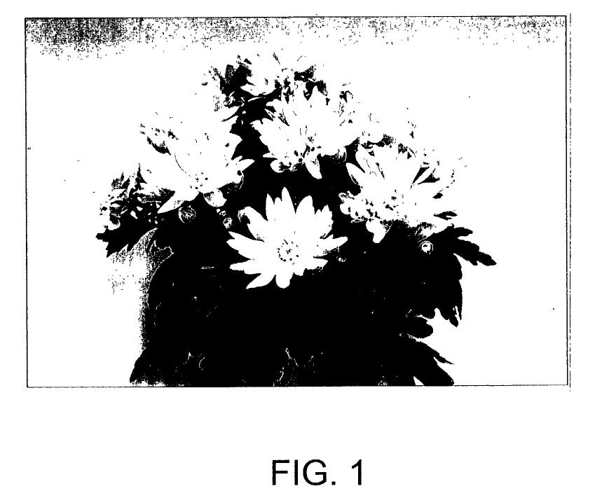

FIG. 2 shows the various stages of bloom of the new cultivar.

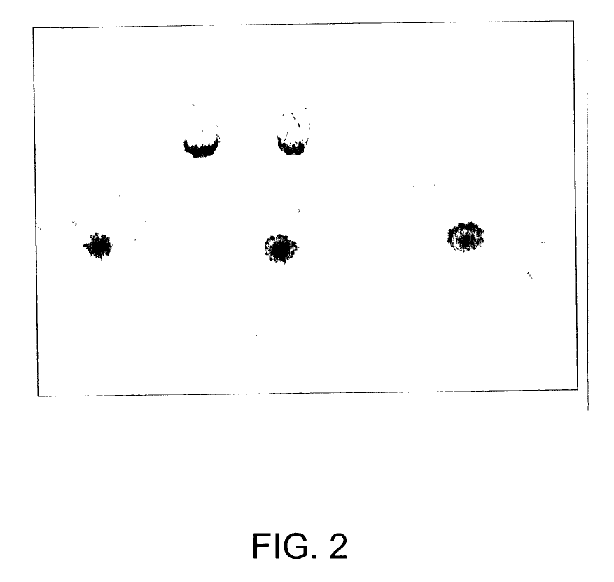

FIG. 3 shows the foliage of the new cultivar

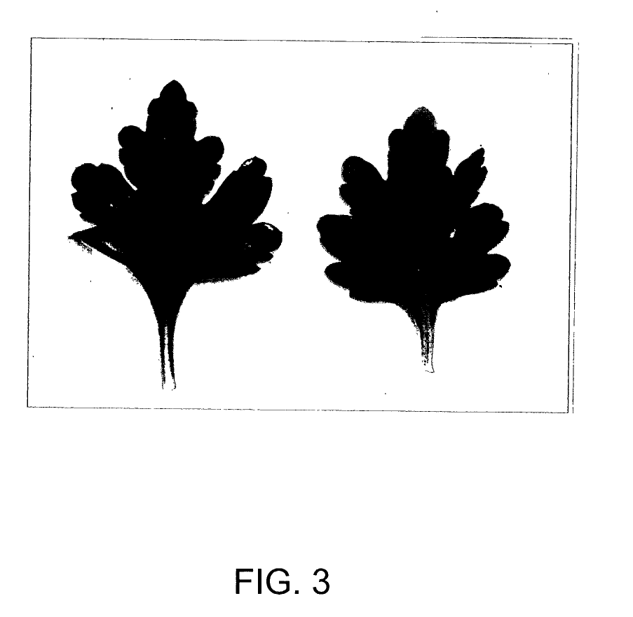

### DESCRIPTION OF THE INVENTION

This new variety of chrysanthemum is of the botanical classification chrysanthemum morifolium. The observations and measurements were gathered from plants grown in a greenhouse in Rijsenhout Holland in a photo-periodic controlled crop under conditions generally used in commercial practice. The greenhouse temperatures during this crop were at day-time between 18° C. and 25° C. and at night 20° C. The photo-periodic response time in this crop was 50 days after an average of eight long days. After this long day period to flowering growth retardants were applied 6 times in an average dose of 1.5 gram/liter water. The plants were observed (directly) during the flowering of this crop. No tests were done on disease or insect resistance or susceptibility. No tests were done on cold or drought tolerance. This new variety produces large sized blooms with huge yellow ray-florets and green disc-florets blooming on the plant for 5 weeks. This new variety of chlysan-themum has been found to retain its distinctive characteristics throughout successive propagations however the phenotype may vary significantly with variations in environment such as light intensity and temperature. To show the phenotype as described ‘POWELL’ can be planted without assimilation lightning (high pressure sodium lamps) between week 50 and week 40 of the next year under greenhouse conditions in Holland. With assimilation lightning (minimum level 2500 lux) it can be planted year-round under greenhouse conditions in Holland.

From the cultivars known to inventor the most similar existing cultivar in comparison to ‘POWELL’ is ‘Miramar’ (U.S. Plant Pat. No. 7469). When ‘Miramar’ and ‘POWELL’ are being compared the following differences are noticed: The difference of ‘Miramar’ and ‘POWELL’ are (1) flower color (2) flower shape and (3) Response time.

The following is a description of the plant and characteristics that distinguish ‘POWELL’ as a new and distinct variety. The color designations are taken from the plant itself. Accordingly, any discrepancies between the color designations and the colors depicted in the photographs are due to photographic tolerances. The color chart used in this description is: The Royal Horticultural Society color chart, edition 1995.

Botanical Description of CULTIVAR ‘POWELL’

Bud

Size.—Large;cross-section 1.4 cm, height 1.2 cm.

Outside Color.—Yellow 13 B.

Involucral bracts.—2 rows, length 7 mm, width 3 mm.

Involucral bracts among disc-florets.—Not present.

Involucral bracts color.—Green 138 B.

Bloom

Type.—Daisy.

Height.—Medium high.

Size.—Large.

Fully Expanded.—8.0-8.5 cm.

Number of blooms per branch.—Approx. 3-4 blooms per branch.

Performance on the plant.—5 weeks.

Seeds.—Produced in large quantities, oval shaped, greybrown 199A, 2 mm in length.

Fragrance.—Typical chrysanthemum.

Color

Center of the flower (disc-florets).—Immature green 143 B. Mature yellow-green 151 A with a small dot of yellow-green 144 B.

Color of upper surface of the ray.—Yellow 5 A florets.

Color of the lower surface of the.—Yellow 6 C ray-florets.

Tonality from Distance.—A pot mum with huge yellow flowers and a green disc.

Color of the surface of the ray.—Yellow 5 B florets after aging of the plant.

Ray florets

Texture.—Upper and under side smooth.

Number.—

Cross-section.—Convex.

Longitudinal axis of majority.—Reflexing and occasionally twisted.

Length of corolla tube.—Short.

Ray-floret length.—3.3-3.7 cm.

Ray-floret width.—1.3-1.5 cm.

Ratio length/width.—Low.

Shape of tip.—Pointed.

Disc florets

Disc diameter.—1.0-1.2 cm.

Distribution of disc florets.—Numerous, clearly visible at all stages of flowering.

Shape.—Tubular.

Color.—Yellow-green 151 A.

Receptacle shape.—Conical raised.

Reproductive Organs

Stamen (present in disc florets only).—Very thin, 3 mm in length.

Number of stamen.—4.

Stamen color.—Green.

Pollen.—No pollen.

Styles (present in both ray and disc.—Thin florets).

Style color.—Yellow-green.

Style.—Length 4 mm.

Stigmas.—Yellow-green.

Stigma Width.—1 mm.

Ovaries.—Enclosed in calyx.

Plant

Form.—A pot mum meant for indoor use.

Growth habit.—Very spreading.

Growth rate.—Moderate.

Height.—26.0-28.0 cm.

Width.—28.0 cm.

Stem Color.—Green 138 B.

Stem Strength.—Very strong.

Stem Brittleness.—Absent.

Stem Anthocyanin.—Coloration Absent.

Length of lateral branch.—From top to bottom 12.0-13.0 cm.

Lateral branch color.—Green 138 B.

Lateral branch, attachment.—Very strong.

Branching (average number of.—Prolific with 6-7 breaks after pinching lateral branches).

Peduncle length.—2.0-3.0 cm.

Peduncle color.—Green 138 B.

Flowering Response(photo-periodic.—50 Days controlled crop, not natural season.

Foliage

Color mature.—Upper side green 137 A. Under side green 138 A.

Color immature.—Upper side green 137 A. Under side green 138 A.

Size.—Large; length 10.5 cm, width 7.0 cm.

Quantity (number per lateral.—5-6 branch).

Shape.—Oval and lobed.

Texture upper side.—Glabrous.

Texture under side.—Pubescent.

Venation arrangement.—Palmate.

Shape of the margin.—Lobed.

Shape of Base of Sinus Between.—Acute.

Lateral Lobes.—

Margin of Sinus Between Lateral.—Mostly diverging, sometimes converging Lobes.

Shape of Base.—Acute sometimes truncate.

Apex.—Cuspidate.

Differences with the comparison varieties

When grown under the same conditions

| | ‘Powell’ | ‘Miramar’ |
|---|---|---|
| Flower color | Yellow 5A | Yellow 7A |
| Flower shape | Ray-florets reflexing | Straight |
| | and occasionally twisted | |
| Response time | 50 days | 55 days |

I claim:

1. A new and distinct variety of chrysanthemum plant as described and illustrated.
